(19) United States
(12) Patent Application Publication
(10) Pub. No.: US 2026/0224738 A1
(43) Pub. Date: Aug. 6, 2026

(54) MICELLE NANOPARTICLES COMPRISING BIOPOLYMER-HEMIN COMPLEX, AND USE THEREOF

(71) Applicant: GWANGJU INSTITUTE OF SCIENCE AND TECHNOLOGY, Gwangju (KR)

(72) Inventors: GI YOONG TAE, Gwangju (KR); ABHISHEK SAHU, Gwangju (KR); KI YOON MIN, Gwangju (KR); SAE HYUN JEON, Gwangju (KR); JUN YOUNG JUNG, Gwangju (KR)

(21) Appl. No.: 19/148,391

(22) PCT Filed: Jan. 8, 2024
(86) PCT No.: PCT/KR2024/000352

§ 371 Date: Jul. 16, 2025

(30) Foreign Application Priority Data
Jan. 16, 2023 (KR) 10-2023-0006192
Jan. 2, 2024 (KR) 10-2024-0000118

(51) Int. Cl. A61K 47/69; A61K 31/555; A61K 47/36; A61P 39/06

(52) U.S. Cl. CPC A61K 47/6931; A61K 31/555; A61K 47/36; A61P 39/06

(57) ABSTRACT

A micelle nanoparticle includes a biopolymer-hemin complex in which a hydrophilic biopolymer and hydrophobic hemin are combined. The nanoparticle has a core-shell structure formed by the self-assembly of the polymer-hemin complex. In the core-shell structure, the hydrophobic hemin forms a core and the hydrophilic biopolymer forms a shell. The micelle nanoparticle has excellent catalase- and superoxide dismutase-like activities, the micelle nanoparticle may effectively treat, alleviate and/or prevent inflammatory diseases.

### BACKGROUND

### 1. Technical Field

The present invention relates to micelle nanoparticles including a biopolymer-hemin complex and uses thereof in the treatment or prevention of inflammatory diseases.

### 2. Background Art

Nanozymes are attracting attention for therapeutic applications due to their high stability and excellent catalytic activity, which allow them to replace natural enzymes that are unstable and expensive. However, most nanozymes are metallic or inorganic materials, and their clinical translation is difficult due to unproven biosafety and limited biodegradability. Hemin is an iron-containing porphyrin that exhibits superoxide dismutase (SOD)-like activity in addition to catalase (CAT)-like activity.

SOD converts highly reactive superoxide anions into hydrogen peroxide, and CAT catalyzes the decomposition of hydrogen peroxide into non-toxic oxygen and water. However, under disease conditions, these cellular ROS scavenging systems cannot fully control ROS levels. Oxidative stress caused by overproduced ROS can lead to cause numerous acute and chronic diseases such as cardiovascular, neurological, renal and immune disorders, as well as cancer. Therefore, alleviating increased oxidative stress and ROS levels is an effective strategy to deal with these diseases, and therapeutic effects of ROS scavenging have been reported in many inflammatory disease models.

Meanwhile, inflammation is an important part of the body's immune response to infection, injury, pain, toxins, or irritation. During the wound healing process, these reactions are critical for eliminating infection. However, if they persist longer than necessary, they may cause harmful effects and lead to chronic inflammation. This persistent chronic inflammatory response, which has detrimental effects on the body and tissues, is known to be mediated through reactive oxygen species (ROS), which are associated with several diseases such as rheumatoid arthritis, atherosclerosis, Alzheimer's disease, inflammatory diseases and cancer. ROS are byproducts of oxygen molecules and tend to degrade oxygen, thus ROS are generated even during respiration. They are naturally highly reactive and can oxidize biologically important molecules such as DNA, proteins, and lipids. ROS act as signaling molecules under physiological conditions, whereas at high concentrations, they act as mediators of inflammation, thus playing a dual role. The human body is constantly attacked by ROS, and in such cases, the human body has an antioxidant defense system to regulate and maintain ROS. When the balance between ROS and defense mechanisms is disrupted, oxidative stress occurs, which plays a key role in the progression of many physiological disorders. Therefore, regulating the excessive production of ROS and balancing oxidative stress can be considered as one of the key strategies for treating inflammation.

Accordingly, the present inventors developed a biopolymer-hemin nanozyme that possesses ROS scavenging ability and is both biocompatible and biodegradable, and confirmed that it can be utilized as a therapeutic agent for diseases caused by excessive reactive oxygen species, such as inflammatory diseases, thus the present invention was completed.

### SUMMARY

An object of the present invention is to provide micelle nanoparticles formed by self-assembly of a polymer-hemin complex in which a biopolymer and hemin are combined.

Another object of the present invention is to provide pharmaceutical and food compositions including the above micelle nanoparticles for the treatment, improvement or prevention of inflammatory diseases.
1. A micelle nanoparticle formed by self-assembly of a polymer-hemin complex in which a hydrophilic biopolymer and hydrophobic hemin are combined.
2. The micelle nanoparticle according to item 1 above, wherein the hydrophilic biopolymer includes any one selected from the group consisting of starch, chitosan, heparin, hyaluronic acid, hemicellulose, lignin, cellulose, chitin, alginate, dextran, pullulan, polyhydroxyalkanoate, fibrin, cyclodextrin, soybean protein, pectin and polylactic acid.
3. The micelle nanoparticle according to item 1 above, wherein the hydrophobic hemin has a structure represented by Formula 1 below:

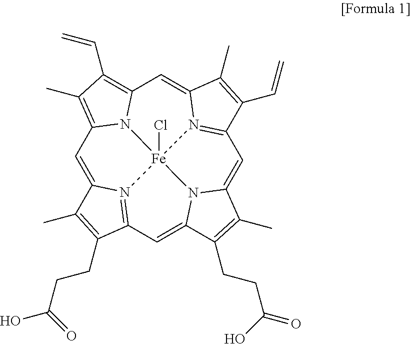

4. The micelle nanoparticle according to item 1 above, wherein the polymer-hemin complex is formed by binding an amine group or thiol group of the hydrophilic biopolymer to a carboxyl group or vinyl group of the hydrophobic hemin.
5. The micelle nanoparticle according to item 1 above, wherein the nanoparticle has a core-shell structure in which the hydrophobic hemin forms a core and the hydrophilic biopolymer forms a shell.
6. The micelle nanoparticle according to item 1 above, wherein a histidine tag is additionally bound to the hydrophilic biopolymer.
7. The micelle nanoparticle according to item 6 above, wherein the histidine tag contains 5 to 20 histidine residues.
8. The micelle nanoparticle according to item 6 above, wherein the hydrophilic biopolymer and histidine tag are contained at a ratio of 1:1 to 1:10.
9. The micelle nanoparticle according to item 1 above, wherein the micelle nanoparticle has a size of 50 to 200 nm.
10. A pharmaceutical composition for the prevention or treatment of inflammatory diseases, including the micelle nanoparticles of any one of items 1 to 9 above.
11. The pharmaceutical composition for the prevention or treatment of inflammatory disease according to item 10 above, wherein the inflammatory disease is any one selected from the group consisting of dermatitis, atopic dermatitis, asthma, conjunctivitis, periodontitis, rhinitis, otitis media, iritis, pharyngitis, tonsillitis, pneumonia, gastric ulcer, pancreatitis, gastritis, ulcerative colitis, Crohn's disease, inflammatory bowel disease, inflammatory cardiovascular disease, myocardial infarction, Alzheimer's disease, diabetic inflammatory disease, colitis, hemorrhoids, gout, ankylosing spondylitis, lupus, fibromyalgia, psoriasis, rheumatoid arthritis, osteoarthritis, osteoporosis, hepatitis, cystitis, nephritis, Sjögren's syndrome and multiple sclerosis.
12. The pharmaceutical composition for the prevention or treatment of inflammatory disease according to item 10 above, wherein the composition is administered by any one route selected from the group consisting of oral administration, inhalation administration, intravenous administration, intramuscular administration, subcutaneous administration, transdermal administration, dermal administration, intrauterine administration, intratumoral administration, and combinations thereof.
13. A food composition for the prevention or improvement of inflammatory diseases, including the micelle nanoparticles of any one of items 1 to 9 above.
14. The food composition for the prevention or improvement of inflammatory diseases according to item 13 above, wherein the inflammatory disease is any one selected from the group consisting of dermatitis, atopic dermatitis, asthma, conjunctivitis, periodontitis, rhinitis, otitis media, iritis, pharyngitis, tonsillitis, pneumonia, gastric ulcer, pancreatitis, gastritis, ulcerative colitis, Crohn's disease, inflammatory bowel disease, inflammatory cardiovascular disease, myocardial infarction, Alzheimer's disease, diabetic inflammatory disease, colitis, hemorrhoids, gout, ankylosing spondylitis, lupus, fibromyalgia, psoriasis, rheumatoid arthritis, osteoarthritis, osteoporosis, hepatitis, cystitis, nephritis, Sjögren's syndrome and multiple sclerosis.
15. The food composition for the prevention or improvement of inflammatory diseases according to item 13 above, wherein the composition is formulated in any one form selected from the group consisting of tablets, capsules, powders, granules, liquids, and pills.

The micelle nanoparticles of the present invention have excellent SOD-mimetic activity and CAT-mimetic activity. The micelle nanoparticles of the present invention exhibit excellent reactive oxygen species scavenging and oxygen generation effects.

The micelle nanoparticles of the present invention exhibit significantly improved cascade catalytic efficiency.

The micelle nanoparticles of the present invention may exhibit therapeutic, ameliorative, or preventive effects on inflammatory diseases due to their antioxidant and anti-inflammatory activities.

### BRIEF DESCRIPTION OF THE DRAWINGS

FIG. 1 illustrates an example of a polymer-hemin complex. CS-H is a chitosan-hemin complex, Hep-H is a heparin-hemin complex, and HA-H is a hyaluronic acid-hemin complex.

FIG. 2A shows the size distribution of CS-H and Hep-H; FIG. 2B shows the zeta potential of CS-H and Hep-H in PBS; and FIG. 2C illustrates the colloidal stability of CS-H and Hep-H in serum-containing medium (#p>0.05, *p<0.05).

FIG. 3A is a graph showing the superoxide anion scavenging activity of Hemin, CS-H, and Hep-H using xanthine/xanthine oxidase; FIG. 3B illustrates the result of observing the stability of SOD activity of Hemin, CS-H, and Hep-H for 14 days in serum condition; FIG. 3C illustrates the result of evaluating the CAT-mimetic activity after monitoring the oxygen generation of Hemin, Hep-H and CS-H by hydrogen peroxide (100 mM); FIG. 3D is a graph showing the result of observing the CAT-mimetic activity of Hemin, CS-H and Hep-H for 14 days in serum condition; FIG. 3E is a graph showing the result of oxygen production through the cascade reaction of Hep-H, CS-H, and Hemin; FIG. 3F is a graph showing the cumulative oxygen generation amount of Hemin, CS-H, and Hep-H for 300 seconds; FIG. 3G is a graph showing the result of evaluating the CAT-mimetic activity after monitoring the oxygen generation of Hemin, Hep-H, and HA-H by hydrogen peroxide (100 mM); and FIG. 3H is a graph showing the superoxide anion scavenging activity of Hemin, Hep-H, and HA-H (#p>0.05, *p<0.05, ** p<0.01, *** p<0.001).

FIG. 4A is a graph showing the cytotoxicity of Hemin, CS-H, and Hep-H on HK-2 cell line after co-culture for 24 hours; FIG. 4B shows the fluorescence image of RITC-labeled CS-H and Hep-H absorbed by HK-2 cells; FIG. 4C is a quantitative graph showing the RITC-labeled CS-H and Hep-H absorbed by HK-2 cells within 24 hours (scale bar=50 μm); FIG. 4D is a graph showing the protective effects of Hemin, CS-H, and Hep-H on HK-2 cell line treated with hydrogen peroxide (200 μM), and FIG. 4E is a graph showing the cytotoxicity of Hemin, CS-H, and Hep-H on HEK293 kidney cells. (#p>0.05, ** p<0.01, *** p<0.001).

FIG. 5A illustrates an AKI mouse model established using glycerol; FIG. 5B is an ex vivo fluorescence image to monitor kidney targeting efficacy and biodistribution of CS-H and Hep-H; and FIG. 5C is a graph showing the biodistribution of CS-H and Hep-H in major organs indicated in total fluorescence intensity (*p<0.05, *** p<0.001).

FIGS. 6A to 6G show: (FIG. 6A) the blood urea nitrogen (BUN) level of mouse serum after treatment of AKI mice with Hemin, CS-H, and Hep-H; (FIG. 6B) the results of creatinine serum level analysis; (FIG. 6C) the kidney microstructure examined by H&E staining results; (FIGS. 6D and 6F) oxidative stress reduction effect of CS-H and Hep-H confirmed by DHE staining results; and (FIGS. 6E and 6G) TUNEL staining images and quantified graphs for histological analysis (scale bar=200 μm) (#p>0.05, * p<0.05, ** p<0.01, *** p<0.001).

FIGS. 7A to 7D show: (FIG. 7A) immunofluorescence staining images of kidneys in AKI mice; and quantitative graphs of KIM-1 (FIGS. 7A and 7C) and MAC387 (FIGS. 7B and 7D) (scale bar=200 μm) (#p>0.05, * p<0.05, *** p<0.001).

FIG. 8 illustrates the amount of histidine H₁₀ bound to Hep-H (#p>0.05, * p<0.05, ** p<0.01, *** p<0.001).

FIG. 9 illustrates the change in surface charge of nanoparticles according to the binding of histidine H₁₀ (#p>0.05, * p<0.05, ** p<0.01, *** p<0.001).

FIGS. 10A to 10C show the results of measuring the sizes and polydispersity index (PDI) values of Hep-H and Hep-H combined with histidine H₁₀ (#p>0.05, * p<0.05, ** p<0.01, *** p<0.001).

FIGS. 11A and 11B illustrate the SOD and CAT activities of nanoparticles combined with histidine H₁₀ (#p>0.05, * p<0.05, ** p<0.01, *** p<0.001).

FIG. 12 illustrates the cascade reaction and oxygen generation amount of nanoparticles combined with histidine H₁₀.

FIGS. 13A to 13C illustrate the superoxide anion removal effect, oxygen generation effect, and a relatively high CAT/SOD activity of Hep-H and Hep-H combined with histidine H₁₀ (#p>0.05, * p<0.05, ** p<0.01, *** p<0.001).

FIGS. 14A and 14B illustrate the cell viability-enhancing effect of Hep-H and Hep-H combined with histidine H₁₀ (#p>0.05, *p<0.05, ** p<0.01, *** p<0.001), wherein

FIG. 14A shows the results of measuring cytotoxicity against the HK-2 cell line after 24 hours of co-culture, and FIG. 14B is a fluorescence image showing RITC-labeled Hep-H and Hep-H combined with histidine H₁₀, which were absorbed by HK-2 cells after 24 hours of co-culture.

FIGS. 15A to 15C illustrate the metabolic activity of Hep-H and Hep-H combined with histidine H₁₀ (#p>0.05, *p<0.05, ** p<0.01, *** p<0.001).

FIG. 16 illustrates the metabolic activity of Hep-H and Hep-H combined with histidine H₁₀ (#p>0.05, *p<0.05, ** p<0.01, *** p<0.001).

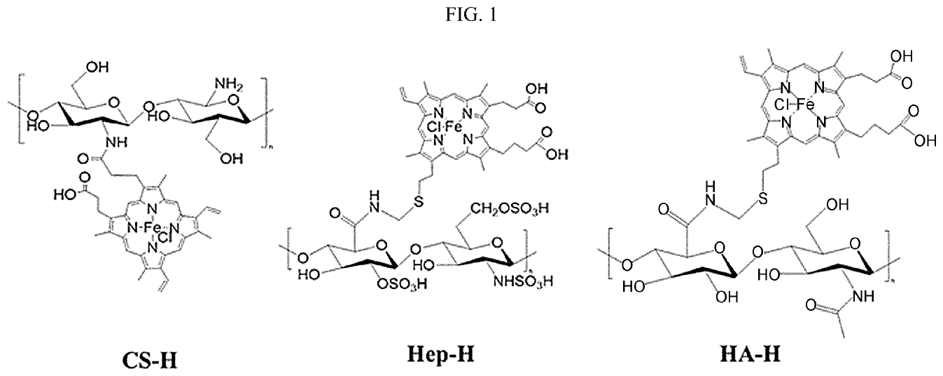

### DETAILED DESCRIPTION

The present invention provides micelle nanoparticles including a biopolymer-hemin complex and uses thereof.

The present invention provides micelle nanoparticles including a biopolymer-hemin complex that has a core-shell structure formed by self-assembly of a polymer-hemin complex in which a hydrophilic biopolymer and hydrophobic hemin are combined, whereby the complex has excellent hydrogen peroxide decomposition enzyme and superoxide dismutase-like activity, as well as effects of treating, improving or preventing inflammatory diseases, and uses thereof.

The present invention provides micelle nanoparticles formed by self-assembly of a polymer-hemin complex in which a hydrophilic biopolymer and hydrophobic hemin are combined. In this case, the hydrophilic biopolymer forms a shell of the micelle, while hydrophobic hemin forms a core of the micelle.

Biopolymer refers to a polymer or a derivative thereof that can be produced by living organisms, which has at least one free amine and/or hydroxyl group in the monomer forming the polymer. For example, biopolymers include starch, chitosan, heparin, hyaluronic acid, hemicellulose, lignin, cellulose, chitin, alginate, dextran, pullulan, polyhydroxyalkanoate, fibrin, cyclodextrin, and proteins (e.g. soybean protein), polysaccharides (e.g., pectin) and/or polylactic acid, etc., but are not limited thereto.

The biopolymer may be modified such that it can bind to the carboxyl or vinyl group of hemin.

The polymer-hemin complex may be formed by binding an amine group or thiol group of the hydrophilic biopolymer to a carboxyl group or vinyl group of the hydrophobic hemin.

In one embodiment, preferably, the hydrophilic biopolymer may be any one selected from the group consisting of chitosan, heparin, and hyaluronic acid. Two or more types of hydrophilic biopolymers may be used in combination.

In one embodiment, when the biopolymer is chitosan, the chitosan-hemin complex may be formed by combining the amine of chitosan and the carboxyl group of hemin.

In one embodiment, when the biopolymer is heparin, the heparin-hemin complex may be formed by binding a thiolated heparin group to a vinyl group of hemin.

In one embodiment, when the biopolymer is hyaluronic acid, the hyaluronic acid-hemin complex may be formed by binding a thiolated hyaluronic acid group to a vinyl group of hemin.

Hemin is an iron-containing porphyrin that has catalase (CAT)-like activity and superoxide dismutase (SOD)-like activity. By conjugating hemin to the biopolymer, a biopolymer-hemin complex with biocompatible and biodegradable properties may be prepared.

The biopolymer-hemin complex forms small and stable nano-aggregates in serum-containing environments and has much more stable SOD and CAT activities compared to hemin, as well as a stepwise cascade reaction (cascade reaction activity) between them. The biopolymer-hemin complex exhibits efficient absorption and cytoprotective effects against reactive oxygen species (ROS) in vitro.

In one embodiment, the hydrophobic hemin may have a structure represented by Formula 1 below:

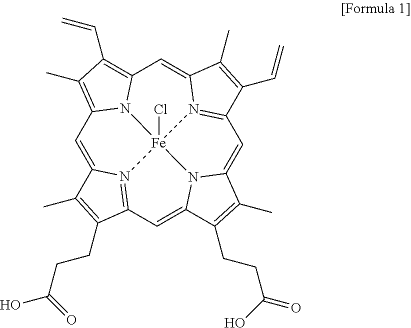

The biopolymer-hemin complex self-assembles to form micelle nanoparticles.

A histidine tag may be additionally bound to the hydrophilic biopolymer of micelle nanoparticles. Due to the binding of the histidine tag, the superoxide dismutase and hydrogen peroxide decomposition enzyme activities of micelle nanoparticles may be increased.

In one embodiment, the histidine tag may include 5 to 20 histidine residues. The number of histidine residues may be 5 to 20, 6 to 19, 7 to 18, 8 to 17, 9 to 16, or 10 to 15.

Micelle nanoparticles and a histidine tag may be combined at a molar ratio of 1:1 to 1:10. For example, the micelle nanoparticles and the histidine tag may be combined at a molar ratio of 1:1 to 1:3, 1:1 to 1:5, 1:2 to 1:5, 1:3 to 1:8, 1:3 to 1:10, and 1:5 to 1:10.

In one embodiment, the micelle nanoparticles may have a size of 50 to 200 nm. The micelle nanoparticles may have a size of 50 to 200 nm, 60 to 180 nm, 70 to 160 nm, 90 to 150 nm, or 100 to 150 nm.

In one embodiment, the micelle nanoparticles have reactive oxygen species (ROS) scavenging ability. The ROS scavenging ability may be due to the activation of superoxide dismutase (SOD) and hydrogen peroxide decomposition enzyme (CAT: catalase).

In one embodiment, the micelle nanoparticles may maintain ROS scavenging ability for at least one week, at least two weeks, at least three weeks, or at least four weeks.

In one embodiment, the nanoparticles may have a low critical micelle concentration (CMC). In a biomimetic environment (containing blood), the heparin-hemin complex has a low critical micelle concentration of 0.01 mg/mL or lower, and the chitosan-hemin complex has a low critical micelle concentration of 0.05 mg/mL or lower. Such a low critical micelle concentration means that the micelle particle shape is effectively maintained even when the concentration of the biopolymer-hemin complex injected into the body and spread throughout the body through the blood is lowered. Therefore, a low micelle critical concentration indicates excellent stability of the biopolymer-hemin complex.

The present invention may provide a method for manufacturing micelle nanoparticles, including the steps of: a) dissolving hemin and a modified biopolymer in a solvent and deionized water, respectively; b) mixing and reacting the hemin solution with the modified biopolymer solution; c) removing unreacted hemin from the mixed solution to form a biopolymer-hemin complex; and d) allowing self-assembly of the hemin residues of the biopolymer-hemin complex by forming hydrophobic interactions.

The method of the present invention may further include the step of obtaining the modified biopolymer by thiolating the biopolymer before step a). In one embodiment, the modified biopolymer may be thiolated heparin or another thiolated biopolymer.

The method of the present invention may further include the step of binding a histidine tag to the micelle nanoparticles after step d). In one embodiment, the histidine tag may include 5 to 20 histidine residues.

The present invention provides a pharmaceutical composition for the prevention or treatment of inflammatory diseases, including micelle nanoparticles obtained by the self-assembly of a biopolymer-hemin complex.

Inflammatory disease may be any one selected from the group consisting of dermatitis, atopic dermatitis, asthma, conjunctivitis, periodontitis, rhinitis, otitis media, iritis, pharyngitis, tonsillitis, pneumonia, gastric ulcer, pancreatitis, gastritis, ulcerative colitis, Crohn's disease, inflammatory bowel disease, inflammatory cardiovascular disease, myocardial infarction, Alzheimer's, diabetic inflammatory disease, colitis, hemorrhoids, gout, ankylosing spondylitis, lupus, fibromyalgia, psoriasis, rheumatoid arthritis, osteoarthritis, osteoporosis, hepatitis, cystitis, nephritis, Sjögren's syndrome and multiple sclerosis.

The pharmaceutical composition of the present invention may include a pharmaceutically acceptable carrier or diluent, and may be formulated in oral dosage forms such as powders, granules, tablets, capsules, suspensions, emulsions, syrups, aerosols, etc., external preparations, suppositories and sterile injectable solutions according to any conventional method.

The pharmaceutically acceptable carriers include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate and mineral oil. Additionally, the pharmaceutical composition may further include diluents or excipients such as fillers, extenders, binders, wetting agents, disintegrants, and surfactants.

Oral solid preparations include tablets, pills, powders, granules, capsules, etc., and these solid preparations may include at least one excipient, such as starch, calcium carbonate, sucrose, or lactose, gelatin, etc., and may include lubricants such as magnesium stearate and talc. Oral liquid preparations include suspensions, oral solutions, emulsions, syrups, etc., and may include diluents such as water and liquid paraffin, humectants, sweeteners, fragrances, and preservatives. Parenteral preparations include sterilized aqueous solutions, non-aqueous solvents, suspensions, emulsions, freeze-dried preparations, and suppositories. Non-aqueous solvents and suspensions include injectable esters such as propylene glycol, polyethylene glycol, vegetable oils such as olive oil, and ethyl oleate, etc. As a base for suppositories, witepsol, macrogol, Tween 61, cacao butter, laurin, glycerogelatin, etc. can be used.

The pharmaceutical composition of the present invention may be administered to mammals such as livestock and humans through various routes, for example, by oral, skin, subcutaneous, muscle, intravenous, intraperitoneal, intrarectal, intrauterine intrathecal or intra-cerebrovascular injection, or topical administration. Accordingly, the composition of the present invention may be formulated in various forms such as tablets, capsules, aqueous solutions, or suspensions. In the case of oral tablets, carriers such as lactose and corn starch and lubricants such as magnesium stearate may usually be added. In the case of capsules for oral administration, lactose and/or dried corn starch may be used as diluents. When an aqueous suspension for oral use is required, the active ingredient may be combined with an emulsifier and/or suspending agent. If necessary, specific sweetening and/or flavoring agents may be added. For intramuscular, intraperitoneal, subcutaneous and intravenous administration, sterile solutions of the active ingredient are usually prepared, and the pH of the solution should be appropriately adjusted and buffered. For intravenous administration, a total concentration of solute should be adjusted to render the formulation isotonic. The composition according to the present invention may be in the form of an aqueous solution including a pharmaceutically acceptable carrier such as saline solution with a pH of 7.4. Aqueous solutions may be introduced into the patient's muscle or bloodstream by local injection.

A dosage of the active ingredient contained in the pharmaceutical composition of the present invention varies depending on the patient's condition and weight, severity of the disease, form of the active ingredient, route and period of administration, and may be appropriately adjusted depending on the patient. For example, the active ingredient may be administered at a dose of 0.0001 to 1000 mg/kg per day, preferably 0.01 to 100 mg/kg, and may be administered once a day or in divided doses. Additionally, the pharmaceutical composition of the present invention may contain the active ingredient in a weight percentage of 0.001 to 90% by weight (“wt %”) based on the total weight of the composition.

The pharmaceutical composition of the present invention may be administered by any one route selected from the group consisting of oral administration, inhalation administration, intravenous administration, intramuscular administration, subcutaneous administration, transdermal administration, dermal administration, intrauterine administration, intratumoral administration, and combinations thereof.

The present invention provides a food composition for the prevention or improvement of inflammatory diseases, including micelle nanoparticles obtained by the self-assembly of a biopolymer-hemin complex.

The food composition of the present invention may be used as a health functional food. Health functional foods refer to foods manufactured and processed using raw materials or ingredients with functionality useful to the human body in accordance with the Health Functional Foods Act. Functionality means ingestion for the purpose of regulating nutrients for the structure and function of the human body, or obtaining useful effects for health care purposes, such as physiological effects.

The food composition of the present invention may include common food additives, and suitability of the additives as a food additive is determined by specifications and standards of the corresponding item in accordance with the general rules and general test methods in the Food Additive Code approved by the Ministry of Food and Drug Safety, unless otherwise specified.

Items listed in the Food Additives Code may include, for example, chemical compounds such as ketones, glycine, potassium citrate, nicotinic acid, cinnamic acid, etc., natural additives such as subsulfuricin, licorice extract, crystalline cellulose, high-quality pigment, guar gum, etc., and mixed preparations such as sodium L-glutamic acid preparations, noodle additive alkaline preparations, preservative preparations, tar coloring preparations, etc.

For the purpose of preventing and/or improving inflammatory diseases, the food composition of the present invention may include 0.01 to 95 wt %, preferably, 1 to 80 wt % of a micelle nanoparticle-containing compound based on the total weight of the composition. Further, for the purpose of preventing and/or improving inflammatory diseases, it may be manufactured and processed in the form of tablets, capsules, powders, granules, liquids, pills, etc.

Hereinafter, the present invention will be described in more detail through examples.

### EXAMPLE

### 1. Examples 1 to 7

1-1. Preparation of ingredients

Water-soluble chitosan (MW 7 kDa) was purchased from Amicogen (Jinju, Korea). Heparin (sodium salt of porcine intestinal mucosa, MW 12 kDa) was purchased from Cellsus Ins (Cincinnati, IA, USA). Hemin, Mn(III) tetrakis(4-benzoic acid) porphyrin chloride (MnTBAP), 1-ethyl-3-(3-dimethylaminopropyl) carbodiimide (EDC), N-hydroxybenzotriazole (HOBt), 1,4-dithiothreitol (DTT), cysteamine, xanthine sodium salt, xanthine oxidase, potassium superoxide, resazurin sodium salt, and rhodamine B isothiocyanate (RITC) were purchased from Sigma Aldrich (St. Louis, MO, USA). WST-1 was purchased from BioMax (Seoul, Korea), and hydrogen peroxide was purchased from Deoksan (Seoul, Korea). Cy5.5-NHS ester was purchased from Lumiprobe (Hunt Valley, MD, USA). Human kidney-2 (HK-2) and human embryonic kidney (HEK293) cell lines were purchased from ATCC (Manassas, VA, USA). Dulbecco's modified eagle's medium (DMEM), Iscove's modified eagle's medium (IMEM), fetal bovine serum (FBS), and antibiotic antifungal agent (AA) for cell culture were purchased from Thermofisher Scientific (Waltham, MA, USA). Human umbilical vein endothelial cells (HUVEC) and endothelial cell basal media (EGM-2) were purchased from Lonza (Basel, Switzerland). ICR mice were purchased from Gbio (Gyeonggi-do, Korea). Blood urea nitrogen (BUN) and creatinine assay kits were purchased from Bioassay Systems (Hayward, CA, USA). Hematoxylin and eosin Y were purchased from BBC Biochemical (Mount Vernon, WA, USA). Dihydroethidium (DHE), terminal deoxynucleotidyl transferase dUTP nick end labeling (TUENL) assay kit, kidney injury molecule-1 (KIM-1) antibody, anti-rabbit IgG (Alexa Fluor 594) and anti-mouse IgG (Alexa Fluor 488) were purchased from Invitrogen (Carlsbad, CA, USA), and MAC387 antibody was purchased from Abcam (Cambridge, UK).

### 1-2. Synthesis of Chitosan-Hemin Conjugate (CS-H)

Chitosan-hemin conjugate (CS-H) was synthesized through EDC coupling reaction. Hemin was completely dissolved in a mixed solvent of pyridine (5 ml) and DMSO (5 ml). HOBt (68 mg) and EDC (58 mg) were added to the hemin solution to activate the carboxylate group of hemin. Then, chitosan (100 mg) dissolved in 2 ml of deionized water was added to the activated hemin, and the mixed solution was reacted at room temperature for 24 hours. The reaction solution was precipitated in acetone to separate the chitosan-hemin conjugate (CS-H), and further dialyzed against deionized water for 2 days to remove any remaining unreacted hemin.

### 1-3. Synthesis of Heparin-Hemin Conjugate (Hep-H)

To prepare the heparin-hemin conjugate (Hep-H), heparin was thiolated using the method described in the prior literature (M. Kim, Y. H. Kim, G. Tae, Acta Biomaterialia 2013, 9, 7833). 200 mg of heparin was dissolved in deionized water, and EDC (111 mg) and HOBt (49.5 mg) were added to the heparin solution. Next, cysteamine (82.5 mg) was reacted with the heparin solution to introduce thiol groups into heparin. This mixed solution was reduced with DTT at pH 8, dialyzed with a 0.1 M NaCl solution containing deionized water for 2 days, and then purified. The reacted heparin was reduced with DTT at pH 8 and purified by dialysis against 0.1 M NaCl containing DIW for 2 days. Next, hemin was conjugated to the thiolated heparin via a Michael-type addition reaction. That is, hemin was first dissolved in 10 mM NaOH, heparin was separately dissolved in deionized water, then the hemin and heparin were mixed, and the pH was set to 8 to 9 for an efficient Michael-type reaction. Afterwards, the mixed solution was reacted for 24 hours and then dialyzed for 2 days. The hemin conjugates (CS-H and Hep-H) were freeze-dried and stored at −20° C. for further experiments.

### 1-4. Synthesis of Hyaluronic Acid-Hemin Complex (HA-H)

To prepare the hyaluronic acid-hemin conjugate (HA-H), thiolated hyaluronic acid was synthesized in the same manner as the thiolated heparin. 200 mg of hyaluronic acid was dissolved in deionized water, and EDC (111 mg) and HOBt (49.5 mg) were added to the hyaluronic acid solution. Next, cysteamine (82.5 mg) was reacted with the hyaluronic acid solution to introduce thiol groups into hyaluronic acid. This mixed solution was reduced with DTT at pH 8, dialyzed with a 0.1 M NaCl solution containing deionized water for 2 days, and then purified. The reacted hyaluronic acid was reduced with DTT at pH 8 and purified by dialysis against 0.1 M NaCl containing deionized water for 2 days. That is, 12 mg hemin was first dissolved in 10 mM NaOH, 50 mg thiolated hyaluronic acid (thiolation degree: 40-42%) was separately dissolved in deionized water, the hemin and hyaluronic acid were mixed, followed by performing an efficient Michael-type reaction. The pH was set to 8.5 to 9. Afterwards, the mixed solution was reacted for 24 hours and then dialyzed for 2 days. The hemin conjugate (HA-H) was freeze-dried and stored at −20° C. for further experiments.

### 1-5. Enzyme-Mimetic Activity and Cascade Reaction of the Biopolymer-Hemin Complex

The superoxide dismutase (SOD)-mimetic activity of hemin and the biopolymer-hemin complex was analyzed by quantifying the remaining superoxide anions generated by the xanthine/xanthine oxidase system.

Samples with the same concentration of hemin were dissolved in PBS (phosphate-buffered saline, containing 10% FBS). A final concentration of 5 μM biopolymer-hemin complex (based on the hemin concentration), xanthine oxidase (0.2 U/ml), xanthine sodium salt (0.1 mg/mL), and WST-1 (0.1 mg/mL) were mixed in PBS to prepare a total volume of 200 μL. The absorbance at 450 nm was measured for 30 minutes using a microplate reader (Varioskan Lux, Thermofisher, Waltham, MA, USA). The absorbance of the control group (a biopolymer-hemin complex or a solution without hemin) was regarded as 100% of the superoxide anion, and the amount of remaining superoxide anion in each sample was calculated by comparison with the control. The catalase (CAT) activity of the biopolymer-hemin complex was evaluated by measuring the amount of oxygen generated through the decomposition of hydrogen peroxide using an oxygen meter (Fire Sting O₂, PyroScience GmbH, Aachen, Germany). Samples with the same final concentration of hemin (5 μM) in PBS containing 10% serum were added to a 5 mM hydrogen peroxide solution, mixed, and the oxygen evolution was measured.

### 1-6. In Vitro Cytotoxicity and Cellular Uptake of the Biopolymer-Hemin Complex

HK-2 and HEK293 kidney cells were cultured in Dulbecco's Modified Eagle's Medium (DMEM) and Improved Minimum Essential Medium (IMEM), respectively, each supplemented with 10% FBS and 1% AA. Cells were seeded at a density of 5,000 cells per well in a 96-well plate and treated with various concentrations of hemin, CS-H, and Hep-H. After 24 hours, cell viability was measured using the Alamar Blue assay. Cellular uptake of the biopolymer-hemin complex was visualized using HK-2 and HUVEC (cultured in EGM-2) cells. RITC-labeled CS-H and Hep-H were added at equivalent fluorescence intensities to the cells. After 24 hours of incubation, cells were observed using a fluorescence microscope (TE2000-U, Nikon, Tokyo, Japan), and the red fluorescence intensity per cell number was calculated using ImageJ software for quantification of cell uptake.

### 1-7. In Vitro Cell Viability Against ROS by Biopolymer-Hemin Complex

HK-2 cells were treated with 200 μM hydrogen peroxide and various concentrations of the biopolymer-hemin complexes. After 24 hours of incubation, cell viability was evaluated using the Alamar Blue assay.

### 1-8. In Vivo Acute Kidney Injury (AKI) Model

All animal experiments were performed in accordance with the Gwangju Institute of Science and Technology Animal Care Committee guidelines (GIST-2021-095). Six-week-old ICR mice (30 g) were used, with free access to food and water. To establish an acute kidney injury (AKI) model, water was restricted for 18 hours. Next, 50% glycerol solution (8 mL/kg) was injected intramuscularly into both hind limbs of each mouse.

### 1-9. Serum Analysis of AKI Mice

After establishing the AKI model, 10 mg/kg of hemin and biopolymer-hemin complex was injected intravenously. Twenty-four hours later, blood was collected for serum urea nitrogen (BUN) and creatinine analysis. Kidneys and other major organs (heart, liver, spleen, and lungs) were also collected for further histological analysis. 1-10. Histological analysis

The kidneys of each group were fixed with 4% formaldehyde, embedded in paraffin, and cut into 5 μm thick sections. H&E staining and TUNEL analysis of kidney sections were performed according to the manufacturer's protocol. For immunostaining of kidney injury molecule-1 (KIM-1) and MAC387, sections were treated with 100-fold diluted KIM-1 and MAC primary antibodies, and 200-fold diluted anti-rabbit IgG (Alexa Fluor 594) and anti-mouse IgG (Alexa Fluor 488) were each used as a secondary antibody. For dihydroethidium (DHE) staining, kidneys were not fixed and freshly inserted into optimal cutting temperature (OCT) compound. Cryo-sectioned kidney sections were treated with 10 μM DHE solution for 30 minutes. Images were analyzed quantitatively using ImageJ software. To observe the long-term biocompatibility of the biopolymer-hemin complex, major organs including the kidneys of rats sacrificed 4 weeks after sample injection were dissected and stained with Hematoxylin & Eosin (H&E).

### 1-11. Statistical Analysis

All statistical analyses were performed using Student's t-test or analysis of variance (ANOVA) using Excel. A p-value of less than 0.05 was considered as a significant difference (#p>0.05 no significant difference, *p<0.05, ** p<0.01, *** p<0.001).

### [Example 1] Synthesis and Size Confirmation of the Biopolymer-Hemin Complex

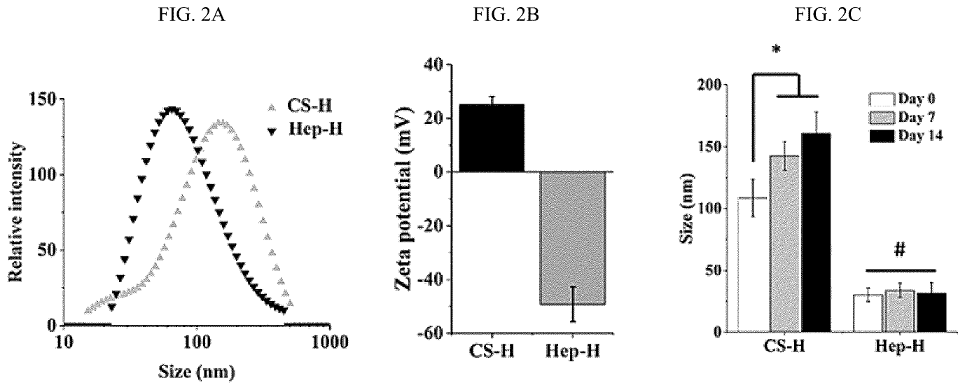

The number of hemin molecules conjugated to the polymer was confirmed at an absorbance of 405 nm (FIGS. 2A to 2C). In CS-H, approximately 1.6 hemins were conjugated to each chitosan, whereas in Hep-H, 3 hemins were conjugated to each heparin. As a result of measurement by DLS, the size of the biopolymer-hemin complex was measured to be 161±12 nm for CS-H and 71±13 nm for Hep-H (FIG. 2A). The positive charge of CS-H was found to support the presence of chitosan outside the nanoparticles, in contrast to the high negative charge of Hep-H due to heparin (FIG. 2B).

To demonstrate that the self-assembly state can be maintained over time, the size of the biopolymer-hemin complex was observed in serum-containing PBS at 37° C. for 2 weeks. An initial size of CS-H in serum was 109±15 nm, slightly smaller than that in PBS, probably due to charge compensation by serum proteins. However, the size of CS-H significantly increased to 143±12 and 161±17 nm after 7 and 14 days, respectively. Meanwhile, the size of Hep-H in serum decreased to 30±5 compared to PBS, and maintained its size over time, showing 34±6 and 32±9 nm after 7 and 14 days, respectively. Because the self-assembled state remained and the size of the enlarged CS-H remained within the nanoscale range after one week, CS-H could continue to be subjected to further experiments. However, Hep-H showed higher colloidal stability in serum than CS-H (FIG. 2C).

### [Example 2] Confirmation of SOD and CAT-Mimetic Activity of the Biopolymer-Hemin Complex

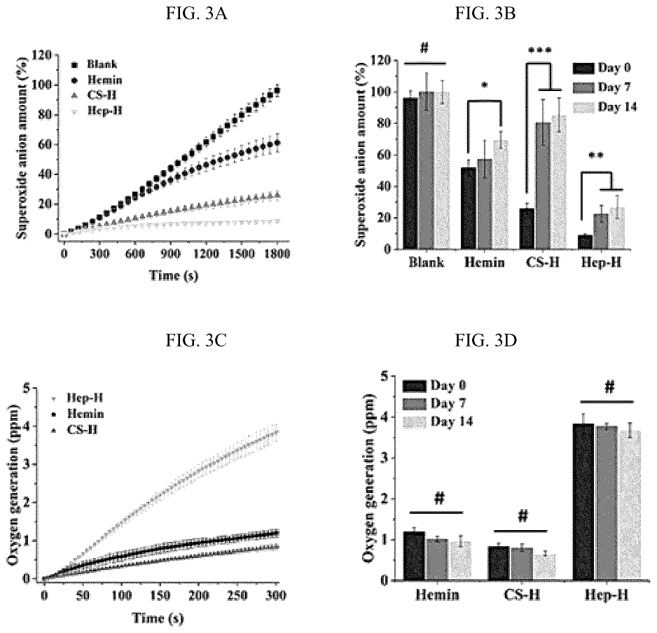

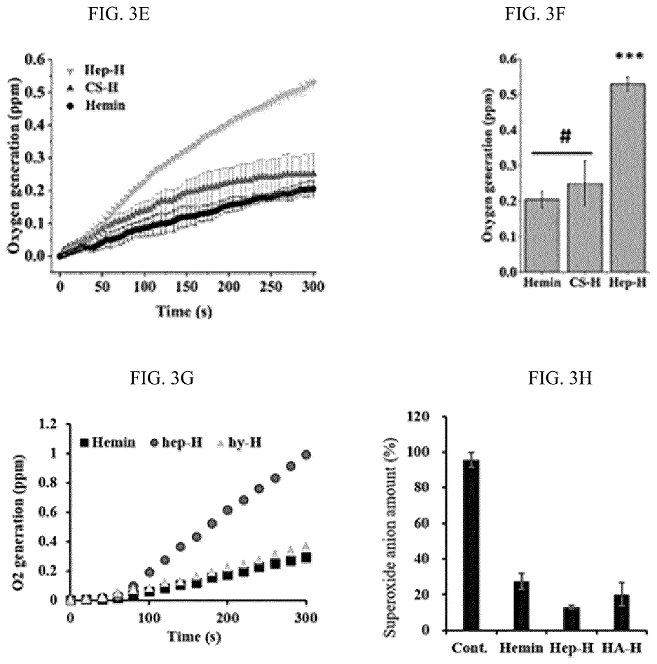

The SOD and CAT-mimetic activities of each biopolymer-hemin complex were analyzed, and the results are shown in FIGS. 3A to 3H. Superoxide anions were generated through the reaction of the xanthine/xanthine oxidase system and detected by a colorimetric method using WST-1 as a substrate. Hemin and biopolymer-hemin were added to the system to observe their ability to decompose superoxide anions. As shown in FIG. 3A, superoxide anion was removed in all groups compared to the control group using PBS instead of hemin. Among them, Hep-H showed the highest scavenging efficiency with more than twice the SOD activity compared to CS-H, and CS-H also had significantly higher SOD activity than unreacted hemin. Referring to FIG. 3H, the SOD-mimetic activity of HA-H was also confirmed. Therefore, the biopolymer-hemin complex was shown to have higher SOD activity compared to free hemin at the same concentration of hemin.

### [Example 3] Confirmation of Stability of SOD Activity of the Biopolymer-Hemin Complex

The stability of SOD activity of the biopolymer-hemin complex was observed for 14 days, and the results are shown in FIGS. 3A to 3H. Referring to FIG. 3B, all groups showed a slight to significant decrease in SOD activity over time (after 7 days and 14 days), compared to the initial state. However, Hep-H maintained much higher SOD activity than unreacted hemin. Therefore, it was confirmed that Hep-H removed 70% or more of superoxide anions even after 14 days. In contrast, the SOD activity of CS-H decreased over time and was lower than that of the unreacted hemin at days 7 and 14 (FIG. 3B). The difference in stability of SOD activity between Hep-H and CS-H is correlated with the colloidal stability of the biopolymer-hemin complex. CAT catalyzes the decomposition of hydrogen peroxide into oxygen and water. Therefore, CAT-mimetic activity was evaluated by monitoring oxygen generation after treating hydrogen peroxide with hemin or biopolymer-hemin complex (FIGS. 3C and 3G).

Similar to SOD activity, Hep-H showed the highest CAT activity, while CS-H and HA-H showed similar CAT activity. All groups showed stable CAT-mimetic activity without significant change over time, in contrast to SOD activity (FIG. 3D). Because hydrogen peroxide (the substrate with CAT activity) is relatively hydrophobic compared to the superoxide anion, the CAT activity of the biopolymer-hemin complex may be less sensitive to the stable dispersion (colloidal stability) of the biopolymer-hemin complex or hemin in an aqueous environment. The higher CAT activity as well as the SOD activity of Hep-H compared to CS-H and HA-H indicated that the charge state of the local environment surrounding the hemin moiety may affect the enzymatic activity of the hemin moiety. Further, since it has both SOD and CAT-mimetic activities, it was found that a cascade reaction between SOD and CAT activities proceeds.

### [Example 4] Confirmation of Cytotoxicity and Cellular Absorption of the Biopolymer-Hemin Complex

The cellular uptake effect of the biopolymer-hemin complex was investigated in vitro using a human proximal tubule cell line (HK-2), which has the ability to reabsorb filtered water, salts, amino acids, and organic solutes.

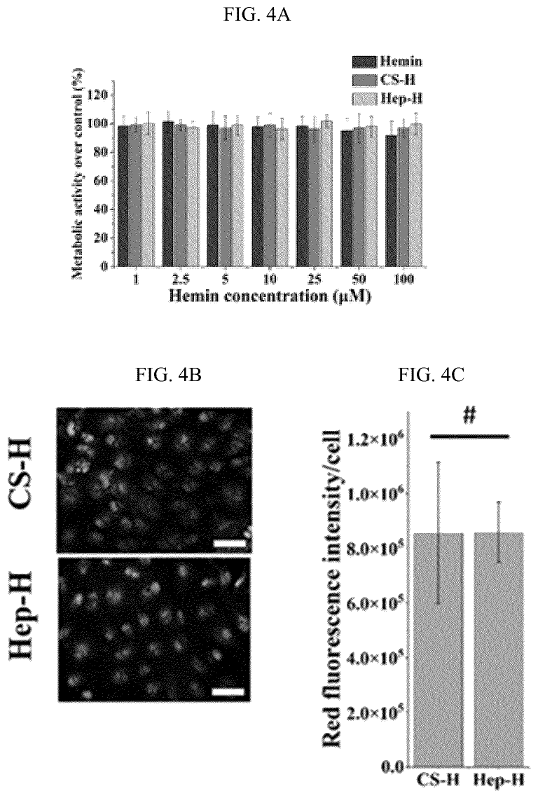

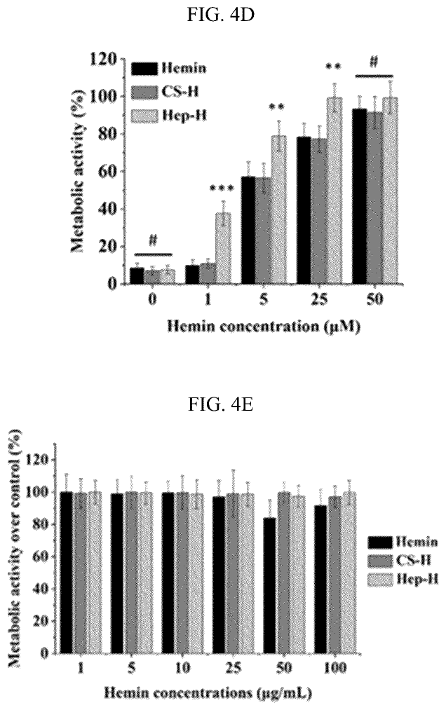

First, the cytotoxicity of the biopolymer-hemin complex was evaluated, and the results are shown in FIGS. 4A to 4E. The biopolymer-hemin complex did not show toxicity up to 100 mM (hemin concentration) in HK-2, as shown in FIG. 4A. Since all materials such as hemin, heparin, and chitosan are biocompatible, the biopolymer-hemin complex was also considered biocompatible. Not only HK-2 but also the human kidney cell line HEK293 showed high cell viability for the biopolymer-hemin complex.

Cellular uptake of the biopolymer-hemin complex into HK-2 was observed using the RITC-labeled complex (FIG. 4B). As shown in FIG. 4B, the biopolymer-hemin complex (red) was distributed in the cytoplasm of the cell. There was no significant difference in fluorescence intensity of the two complexes (Hep-H and CS-H) per cell (FIG. 4C). As such, it could be confirmed that chitosan was well absorbed into the proximal tubule in vitro, and it also could be confirmed that chitosan generally induces cellular uptake due to positive charge and appropriate hydrophobicity.

### [Example 5] Confirmation of Cytoprotective Effect of the Biopolymer-Hemin Complex Against ROS

It was confirmed that the biopolymer-hemin complex removes ROS such as hydrogen peroxide and protects cells from oxidative stress (FIG. 4D). The ROS protective effect of the biopolymer-hemin complex was assayed in HK-2 cells with hydrogen peroxide. Various concentrations of hemin and biopolymer-hemin complexes with the same amount of hemin were added to cells treated with hydrogen peroxide (200 μM). The survival rate of cells was improved by increasing the concentration of the biopolymer-hemin complex or hemin. The control group, which was not treated with any sample, showed a cell survival rate of less than 10%. However, when the hemin concentration was 1 μM, the cell viability against hydrogen peroxide increased dramatically only for Hep-H, whereas hemin and CS-H showed no protective effect at all. CS-H and hemin showed improved protective effects at higher concentrations (5 and 25 μM), but Hep-H still showed higher protective effects than hemin and CS-H. When the hemin concentration was sufficiently high (50 μM), all groups showed excellent protective effect against 200 μM hydrogen peroxide. Therefore, Hep-H had the highest cytoprotective effect against hydrogen peroxide, with no significant difference observed between CS-H and hemin. These results are consistent with the higher CAT activity of Hep-H and the similar CAT activity between CS-H and hemin (FIGS. 3C and 3D).

The excellent cytoprotective effect of especially Hep-H among biopolymer-hemin complexes against oxidative environments was confirmed in vitro.

### [Example 6] Confirmation of In Vivo Kidney Targeting Efficacy and Biodistribution of the Biopolymer-Hemin Complex

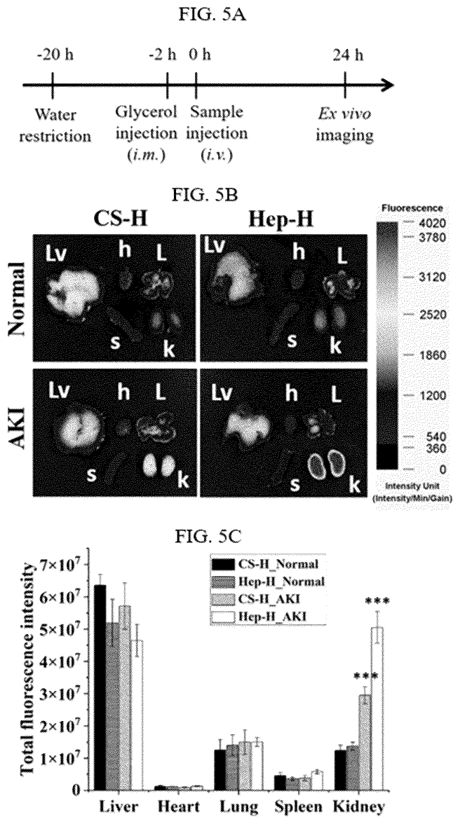

An AKI mouse model was established using glycerol (FIG. 5A). Cy5.5-labeled biopolymer-hemin complex was injected intravenously into normal and AKI mice, and the mice were sacrificed 24 hours later. Major organs were extracted to monitor the kidney-targeting efficacy and biodistribution of the biopolymer-hemin complex. In normal mice, there was no noticeable difference in biodistribution between CS-H and Hep-H (FIGS. 5B and 5C). Further, no signs of renal targeting were observed. However, in AKI mice, a marked increase in signal was observed in the kidney for both CS-H and Hep-H compared to normal mice. Hep-H showed a much higher fluorescence signal than CS-H in the kidney. Except for the kidney, the biodistribution of other organs was similar in all groups (between CS-H and Hep-H, and between normal and AKI mice). Hep-H showed slightly lower signal in the liver than CS-H in both normal and AKI mice, and AKI mice showed slightly lower signal in the liver than normal mice for both CS-H and Hep-H. The increased accumulation of the biopolymer-hemin complexes in the kidneys of AKI mice compared with normal mice indicated that biopolymer-hemin complexes can selectively target the damaged kidneys rather than normal kidneys. The different targeting efficiencies of the biopolymer-hemin complexes were found to be relevant to the anatomical structure of the kidney.

In conclusion, the biopolymer-hemin complex could be selectively delivered and accumulated in the kidneys of AKI mice in one day, and Hep-H is confirmed to be much more efficient in kidney-targeted delivery than CS-H.

### [Example 7] Confirmation of the Therapeutic Effect of the Biopolymer-Hemin Complex on AKI In Vivo

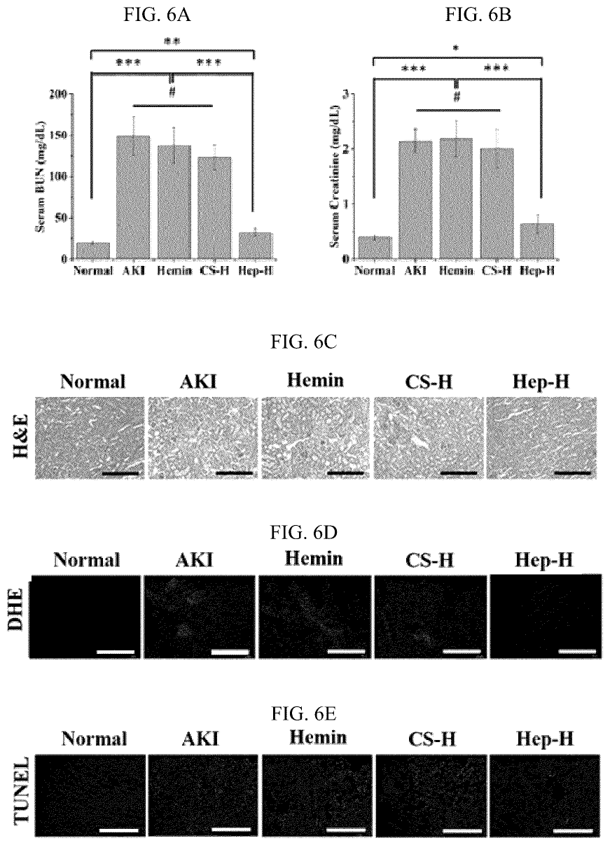

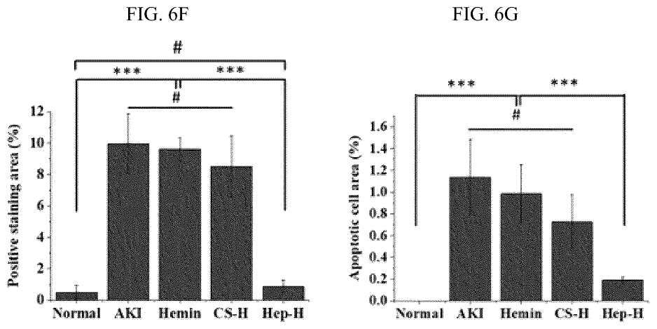

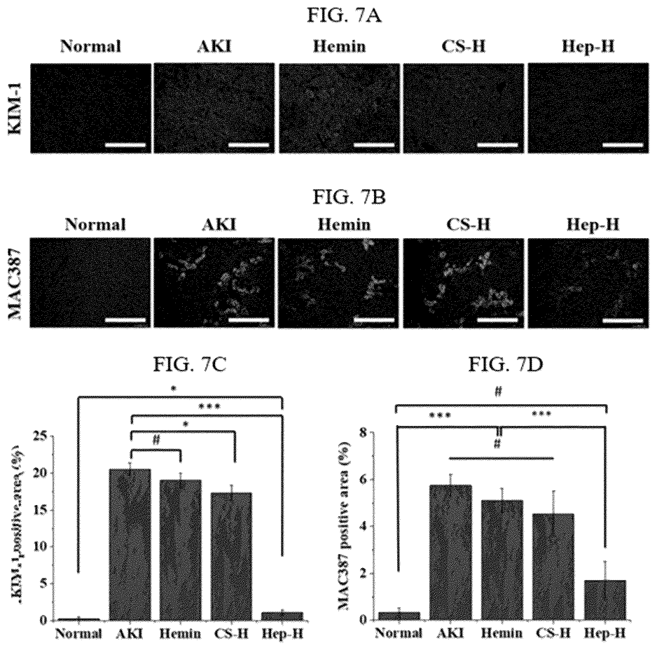

The protective effect of the biopolymer-hemin complex on AKI was evaluated by serum and histological analysis, and the results are shown in FIGS. 6A to 7D. Serum levels of blood urea nitrogen (BUN) and creatinine, which are common indicators of renal dysfunction, were analyzed. Both groups showed similar trends, as shown in FIGS. 7A and 7B. Compared with normal mice, AKI mice had approximately four-fold increases in BUN and creatinine levels, confirming their successful preparation as an AKI model. Administration of hemin to AKI mice did not induce significant changes in BUN and creatinine levels compared to AKI mice. Although significant levels of kidney-targeted delivery were detected, CS-H had very similar effects to hemin and had no therapeutic effect in terms of serum analysis. In contrast, Hep-H showed excellent therapeutic effect in lowering BUN and creatinine levels to ⅓ of AKI mouse levels and very close to normal mouse levels.

Histological analysis of kidneys also demonstrated the therapeutic effect of Hep-H on AKI mice. Kidney microstructure was observed through H&E staining images (FIG. 6C). Compared with the dense tissue with intact glomeruli in normal kidneys, AKI kidneys showed sparse tissue with damaged glomeruli, once again confirming the successful establishment of the AKI model. Both hemin and CS-H groups showed similar histological characteristics of AKI mice, confirming that there was limited therapeutic effect of hemin or CS-H administration. In contrast, the kidney structure in the Hep-H group showed histological morphology very similar to the normal kidney. A dense tissue structure with slightly damaged glomeruli that were smaller than normal kidneys and slightly irregular in shape was observed. Therefore, histological analysis also supports the very potent and efficient therapeutic effect of Hep-H on AKI compared to the lack of noticeable effect of CS-H or hemin.

Tissue ROS were directly visualized using dihydroethidium (DHE) staining to confirm the effect of the biopolymer-hemin complex in alleviating inflammation by reducing oxidative stress (FIGS. 6D and 6F). In contrast to no detectable ROS in normal kidneys, significantly stronger ROS levels were detected in AKI mice. Based on the above analysis, intravenous administration of hemin or CS-H was not effective in reducing ROS levels in the damaged kidney. In contrast, Hep-H almost completely alleviated ROS signaling in the kidney, which was consistent with other analyses. Oxidative stress from ROS may damage tissues/organs and even induce cell death.

Referring to FIGS. 7A and 7C, immunostaining of kidney injury molecule-1 (KIM-1), which is another important biomarker for kidney injury, showed similar expression of KIM-1 for both the AKI group and the hemin group. The CS-H group showed a slightly reduced KIM-1 signal, but KIM-1 expression in the Hep-H group was significantly lower than that in the AKI group. Macrophages were also stained as a representative marker for inflammation (FIGS. 7B and 7D). Similar to KIM-1 immunostaining, strong macrophage signals were observed in the AKI group, hemin group and CS-H group, and there was no statistical difference. However, the Hep-H group also showed a significant decrease in macrophage signaling. Therefore, all analyses demonstrated a very strong therapeutic effect of Hep-H by intravenous administration on AKI, and it was shown efficient removal of ROS leading to reduced inflammation (macrophage migration), which can effectively prevent structural and functional damage to the kidney.

In contrast, the therapeutic effect of CS-H was minimal despite significant delivery of CS-H to the kidney (FIG. 5C). The limited therapeutic effect of CS-H compared to Hep-H suggests that exogenously delivered SOD activity alone is not sufficient to reduce overall oxidative stress caused by ROS in inflammatory damage in vivo. As previously systematically analyzed using SOD and CAT enzymes, the cascade activity of SOD/CAT is important for efficient therapeutic outcome.

The biocompatibility of the biopolymer-hemin complex was evaluated by intravenous injection of the biopolymer-hemin complex into normal mice. After 4 weeks, the animals were sacrificed and major organs (heart, liver, spleen, lungs and kidneys) were extracted. Each organ was histologically analyzed by H&E staining as shown in FIGS. 6A to 6G. All major organs showed no difference in microstructure compared to normal mice injected with PBS, confirming the biocompatibility and biosafety of the biopolymer-hemin complex.

### 2. Examples 8 to 11

2-1. Preparation of Ingredients

Hemin, xanthine, xanthine oxidase, dihydroethidium (DHE) and rhodamine B isothiocyanate (RITC) were purchased from Sigma Aldrich (St. Louis, MO, USA). Histidine 10-mer (H₁₀) was purchased from Synpeptide (Beicai, Shanghai, China). WST-1 was purchased from BioMax (Seoul, Korea), and hydrogen peroxide was purchased from Deoksan (Seoul, Korea). Micro BCA protein quantification kit, Dulbecco's modified eagle's medium (DMEM), fetal bovine serum (FBS), and antibiotic antifungal agent (AA) for cell culture were purchased from Thermofisher Scientific (Walthan, MA, USA). Human kidney-2 (HK-2) cell line was purchased from ATCC (Manassas, VA, USA).

### 2-2. Preparation of Heparin-Hemin-Histidine Complex

The heparin-hemin conjugate and histidine were made by physical interaction through electrostatic attraction of heparin and histidine. Heparin-hemin (1.7 mg) was completely dissolved in PBS (phosphate-buffered saline) (1 mL). 0.2 mg, 0.4 mg, or 1 mg of histidine was completely dissolved in PBS (1 mL) (heparin-hemin: histidine=1:1, 1:2, and 1:5). The two solutions were mixed, and 8 mL of PBS was added, followed by a reaction at room temperature for 24 hours. The reaction solution was dialyzed in deionized water for 2 days to remove unattached histidine. Afterwards, the mixture was filtered using a 0.2 μm syringe filter, freeze-dried, and stored at −20° C. for further experiments.

### 2-3. Enzyme-Mimetic Activity and Cascade Reaction of Heparin-Hemin-Histidine Complex

The superoxide dismutase (SOD)-mimetic activity of hemin, heparin-hemin, and heparin-hemin-histidine complexes was analyzed by quantifying the remaining superoxide anions generated by the xanthine/xanthine oxidase system.

Samples with the same concentration of hemin were dissolved in PBS. Heparin-hemin-histidine complex (based on the hemin concentration) at a final concentration of 5 μM, xanthine oxidase (0.2 U/mL), xanthine sodium salt (0.1 mg/mL), and WST-1 (0.1 mg/mL) were mixed in PBS to make a total volume of 200 μL. A change in absorbance at 450 nm was measured for 30 minutes using a microplate reader (Varioskan Lux, Thermofisher, Waltham, MA, USA). The absorbance of the control (a solution without hemin) was regarded as 100% of the superoxide anion, and the amount of the remaining superoxide anion in each sample was calculated by comparing with the control. The hydrogen peroxide decomposition enzyme (CAT) activity of the heparin-hemin-histidine complex was evaluated by measuring the amount of oxygen generated through the decomposition of hydrogen peroxide using an oxygen meter (Fire Sting O₂, PyroScience GmbH, Aachen, Germany). A heparin-hemin-histidine complex was added to make a final concentration of 5 μM and mixed with hydrogen peroxide (5 mM), followed by measuring the amount of oxygen generated in PBS.

### 2-4. In Vitro Cytotoxicity and Cellular Uptake of Heparin-Hemin-Histidine Complex

HK-2 cells were cultured in DMEM medium supplemented with 10% FBS and 1% AA. 10,000 cells were seeded in each well of a 96-well plate and treated with 50, 100, 150, and 200 μM heparin-hemin and heparin-hemin-histidine complexes. After 24 hours, cell viability was measured using the Alamar Blue assay. Cellular uptake of the heparin-hemin-histidine complex was visualized in HK-2 cells. RITC-labeled heparin-hemin and heparin-hemin-histidine complexes were added at equivalent fluorescence intensities to the cells. After 24 hours of incubation, the cells were observed using a fluorescence microscope (TE2000-U, Nikon, Tokyo, Japan), and the red fluorescence intensity per cell was calculated using ImageJ software to quantify the complexes absorbed into the cells.

### 2-5. Cell Viability Against ROS Via Heparin-Hemin-Histidine Complex In Vitro

DHE was used to visualize intracellular superoxide. HK-2 cells were seeded at a density of 10,000 cells per well in a 96-well plate and treated with xanthine (200 μM) and xanthine oxidase (1 mU). After culturing for 24 hours, the cells were washed with PBS, treated with DHE (10 μM), and incubated for 30 minutes. After removing the supernatant from each well, the cells were washed three times with PBS and observed using a fluorescence microscope (TE2000-U, Nikon, Tokyo, Japan). To quantify superoxide, green fluorescence intensity was calculated using ImageJ software. The cell viability was determined using the Alamar Blue assay on cells treated in the same manner as above.

To confirm the protective effect of the heparin-hemin-histidine complex against hydrogen peroxide, HK-2 cells were seeded at a density of 5,000 cells per well in a 96-well plate. Cells were treated with 200 μM hydrogen peroxide and hemin, heparin-hemin or heparin-hemin-histidine complex. After 24 hours of incubation, cell viability was evaluated using the Alamar Blue assay.

### 2-6. Statistical Analysis

All statistical analyses were performed using Student's t-test or ANOVA (analysis of variance) using Excel. A p-value of less than 0.05 was considered as a significant difference (#p>0.05, *p<0.05, ** p<0.01, *** p<0.001).

### [Example 8] Production and Size Confirmation of Heparin-Hemin-Histidine Complex

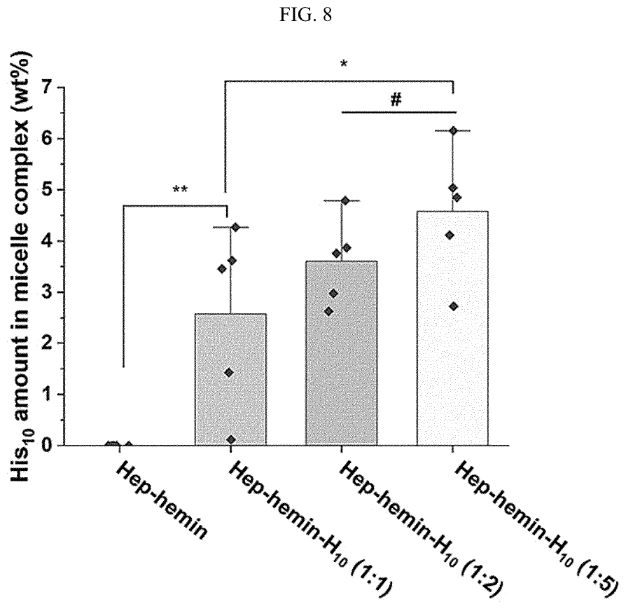

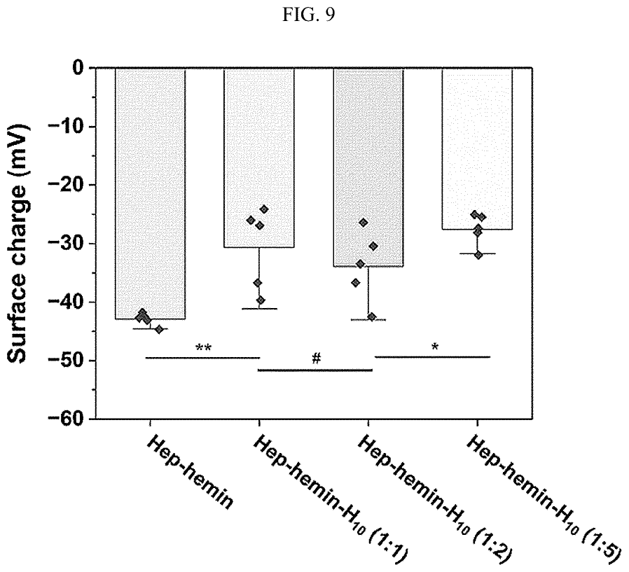

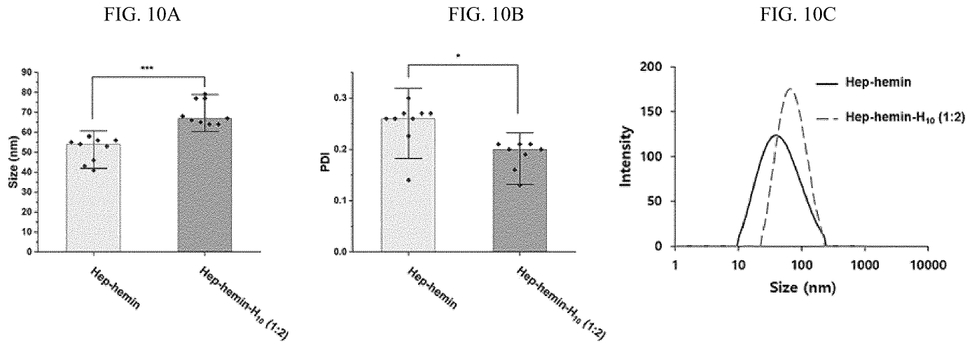

The mixed histidine was quantified using the micro BCA protein quantification method (FIG. 8). As the amount of added increased, an amount of histidine incorporated into the complex also increased. The surface charge increased when histidine was included, thereby supporting the inclusion of positively charged histidine (FIG. 9). As a result of measurement by DLS, the size of heparin-hemin-histidine was 66±11 nm at 1:1, 79±12 nm at 1:2, and 100±17 nm at 1:5 (Table 1 and FIGS. 10A to 10C).

TABLE 1

| Ratio of Hep-hemin to H₁₀ | Size | PDI |
|---|---|---|
| Without H10 | 59 ± 4 | 0.26 ± 0.02 |
| 1:1 | 66 ± 11 | 0.26 ± 0.02 |
| 1:2 | 79 ± 12 | 0.25 ± 0.01 |
| 1:5 | 100 ± 17 | 0.16 ± 0.03 |

### [Example 9] Confirmation of SOD and CAT-Mimetic Activity of Heparin-Hemin-Histidine Complex

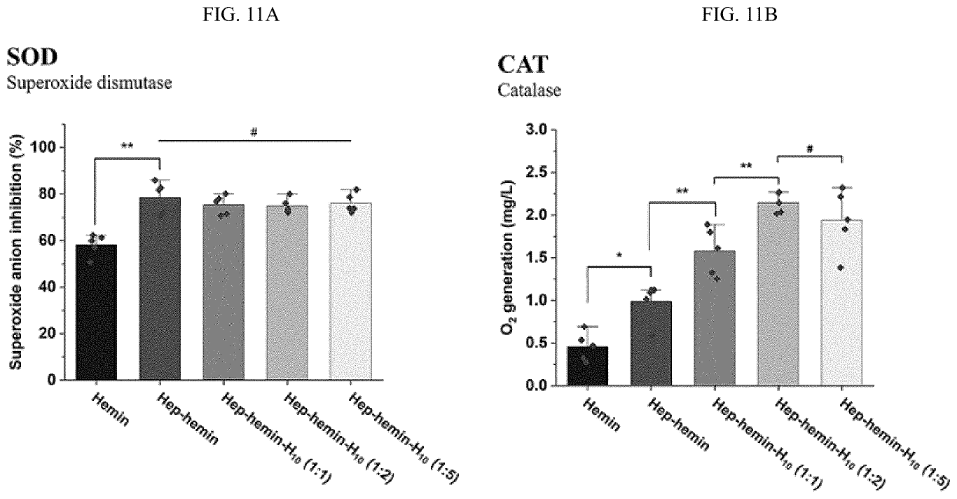

The SOD and CAT-mimetic activities of the heparin-hemin-histidine complex were analyzed, and the results are shown in FIGS. 11A and 11B. When superoxide anions were generated through the reaction of the xanthine/xanthine oxidase system, they were detected by a colorimetric method using WST-1 as a substrate. Hemin and heparin-hemin complex were added to the system to observe the superoxide anion decomposition ability. As can be seen in FIG. 11A, the removal of superoxide anions increased in heparin-hemin and heparin-hemin-histidine complexes compared to hemin, but histidine had no additional effect.

CAT catalyzes the decomposition of hydrogen peroxide into oxygen and water. Therefore, CAT-mimetic activity was evaluated by measuring oxygen generation after treating hydrogen peroxide with hemin, heparin-hemin, or heparin-hemin-histidine complex (FIG. 11B). Unlike SOD activity, CAT activity in the presence of histidine, increased compared to heparin-hemin. The heparin-hemin and histidine produced at a ratio of 1:2 showed the highest CAT activity, and a ratio of 1:5 also showed similar CAT activity. These results provide evidence supporting that histidine, known as an acid-base catalyst, affected CAT activity.

Further, as the complex exhibited both SOD- and CAT-mimetic activities, a cascade reaction between SOD and CAT activities was confirmed (FIG. 12).

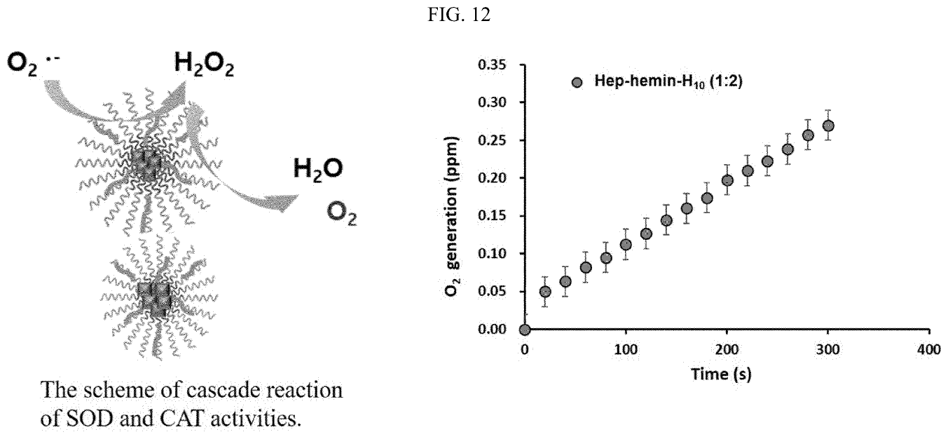

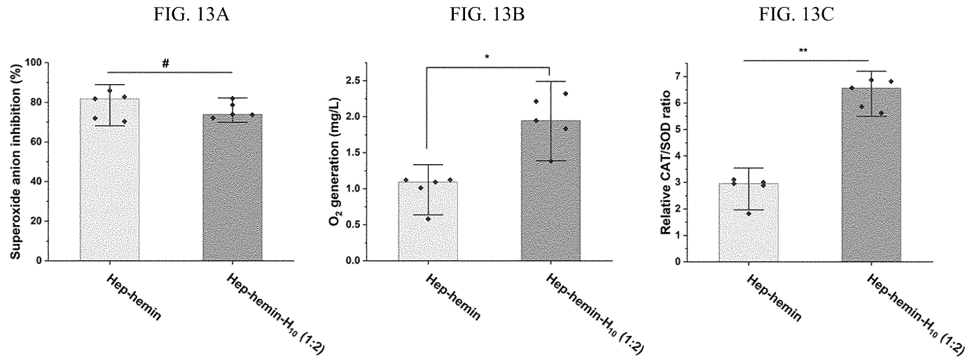

Further, it was confirmed that the heparin-hemin-histidine complex showed improved oxygen generation effect and a relatively high CAT/SOD activity compared to the heparin-hemin complex (FIGS. 13A to 13C).

### [Example 10] Confirmation of Cytotoxicity and Cellular Absorption of Heparin-Hemin-Histidine Complex

The cytotoxicity and cellular uptake effects of heparin-hemin and heparin-hemin-histidine complexes were investigated in vitro using a human proximal tubule cell line (HK-2), which has the ability to reabsorb filtered water, salts, amino acids, and organic solutes.

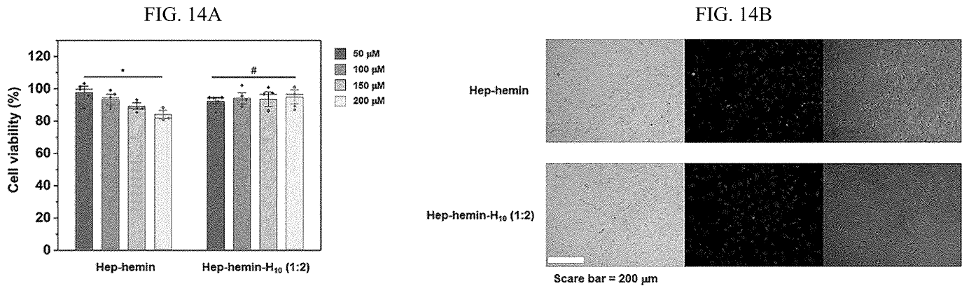

First, the cytotoxicity of heparin-hemin and heparin-hemin-histidine complexes was evaluated, and the results are shown in FIG. 14A. Either heparin-hemin or heparin-hemin-histidine complex showed no toxicity up to 200 μM (based on the hemin concentration), as shown in FIG. 14A. All of the heparin, hemin, and histidine exhibited excellent biocompatibility, and the complex was also confirmed to be highly biocompatible.

Cellular uptake of heparin-hemin and heparin-hemin-histidine complexes into HK-2 was observed using the RITC-labeled complexes. As shown in FIG. 14B, the heparin-hemin-histidine complex showed slightly less cellular uptake than heparin-hemin. However, the extent of cellular uptake was less than 10%, and the heparin-hemin-histidine complex was positioned in the cytoplasm of most cells. In this way, it was confirmed that both heparin-hemin and heparin-hemin-histidine complexes were well absorbed into the proximal tubules in vitro, and although histidine had an effect on cellular uptake, it was confirmed that the effect thereof was minimal.

### [Example 11] Confirmation of Cytoprotective Effect of Heparin-Hemin-Histidine Complex Against ROS

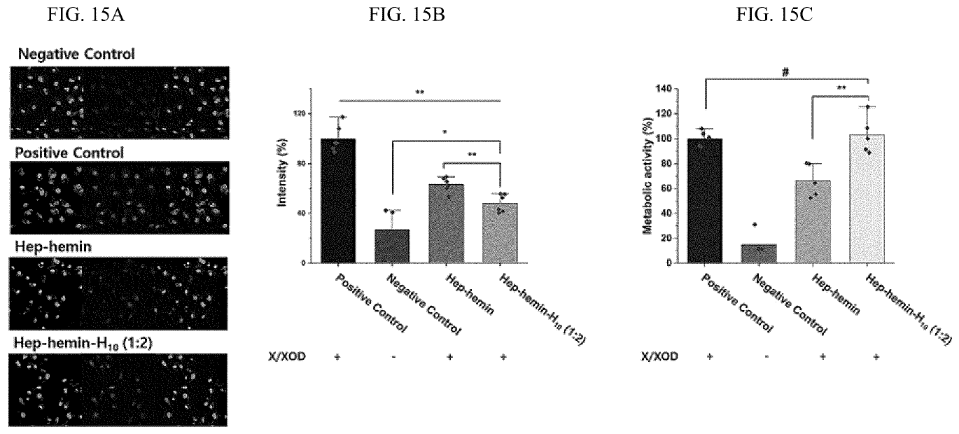

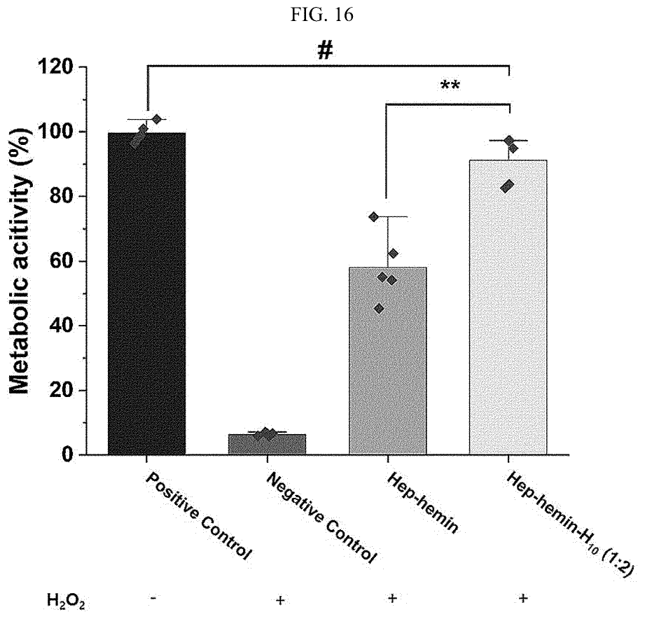

It was confirmed that the heparin-hemin-histidine complex removed ROS such as superoxide anion and hydrogen peroxide, and protected cells from oxidative stress (FIGS. 15A to 16). The ROS protective effect of the heparin-hemin-histidine complex was analyzed by treating HK-2 cells with xanthine, xanthine oxidase or hydrogen peroxide. After treating HK-2 cells with xanthine and xanthine oxidase to generate superoxide anions, a heparin-hemin-histidine complex with the same amount of hemin as the heparin-hemin complex was added to the cells, and the effect was confirmed 24 hours later. DHE staining was used to visualize the remaining superoxide anion.

As can be seen in FIGS. 15A to 15C, when the red fluorescence signal of the remaining superoxide anion was quantified, the heparin-hemin-histidine complex showed a better superoxide anion suppression effect than the heparin-hemin complex.

Hydrogen peroxide (200 μM) and heparin-hemin, as well as a heparin-hemin-histidine complex with the same amount of hemin, were added to the cells (FIG. 16). A cell survival rate was the highest when the heparin-hemin-histidine complex was added. The control group without treatment using the sample showed a cell viability of less than 10%, while the heparin-hemin complex showed a cell viability of approximately 60%, and the heparin-hemin-histidine complex showed a cell viability of approximately 90% or more. These results are consistent with the higher CAT activity of heparin-hemin-histidine.

1. A micelle nanoparticle formed by self-assembly of a polymer-hemin complex in which a hydrophilic biopolymer and hydrophobic hemin are combined.

2. The micelle nanoparticle according to claim 1, wherein the hydrophilic biopolymer comprises any one selected from the group consisting of starch, chitosan, heparin, hyaluronic acid, hemicellulose, lignin, cellulose, chitin, alginate, dextran, pullulan, polyhydroxyalkanoate, fibrin, cyclodextrin, soybean protein, pectin and polylactic acid.

3. The micelle nanoparticle according to claim 1, wherein the hydrophobic hemin has a structure represented by Formula 1 below:

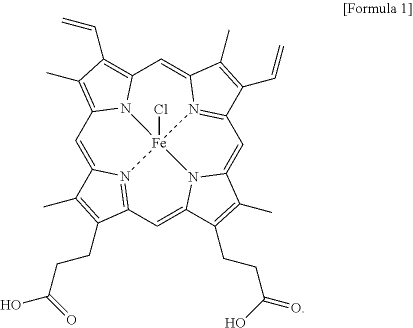

4. The micelle nanoparticle according to claim 1, wherein the polymer-hemin complex is formed by binding an amine group or thiol group of the hydrophilic biopolymer to a carboxyl group or vinyl group of the hydrophobic hemin.

5. The micelle nanoparticle according to claim 1, wherein the nanoparticle has a core-shell structure in which the hydrophobic hemin forms a core and the hydrophilic biopolymer forms a shell.

6. The micelle nanoparticle according to claim 1, wherein a histidine tag is additionally bound to the hydrophilic biopolymer.

7. The micelle nanoparticle according to claim 6, wherein the histidine tag comprises 5 to 20 histidine residues.

8. The micelle nanoparticle according to claim 6, wherein the hydrophilic biopolymer and histidine tag are contained at a ratio of 1:1 to 1:10.

9. The micelle nanoparticle according to claim 1, wherein the micelle nanoparticle has a size of 50 to 200 nm.

10. A pharmaceutical composition comprising the micelle nanoparticle of claim 1, and a pharmaceutically acceptable carrier and/or diluent.

11. (canceled)

12. (canceled)

13. A food composition comprising the micelle nanoparticle of claim 1, wherein the food composition is formulated in a form selected from the group consisting of a tablets, a capsule, powders, a granule, a liquid, and a pill.

14. (canceled)

15. (canceled)

16. A method for treatment of an inflammatory disease, the method comprising:
administering a composition comprising the micelle nanoparticle to a subject in need thereof.

17. The method of claim 16, wherein the inflammatory disease is selected from the group consisting of dermatitis, atopic dermatitis, asthma, conjunctivitis, periodontitis, rhinitis, otitis media, iritis, pharyngitis, tonsillitis, pneumonia, gastric ulcer, pancreatitis, gastritis, ulcerative colitis, Crohn's disease, inflammatory bowel disease, inflammatory cardiovascular disease, myocardial infarction, Alzheimer's disease, diabetic inflammatory disease, colitis, hemorrhoids, gout, ankylosing spondylitis, lupus, fibromyalgia, psoriasis, rheumatoid arthritis, osteoarthritis, osteoporosis, hepatitis, cystitis, nephritis, Sjögren's syndrome, multiple sclerosis, and a combination thereof.

18. The method of claim 16, wherein the composition is administered by a route selected from the group consisting of oral administration, inhalation administration, intravenous administration, intramuscular administration, subcutaneous administration, transdermal administration, dermal administration, intrauterine administration, intratumoral administration, and a combinations thereof.
